(19) United States
(12) Patent Application Publication
(10) Pub. No.: US 2008/0008669 A1
(43) Pub. Date: Jan. 10, 2008

(54) Photostable sunscreen compositions comprising cinnamate ester UV-B filters and s-triazine compounds

(75) Inventors: Herve Richard, Les Pavillons Sous Bois (FR); Didier Candau, Bievres (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 11/702,167

(22) Filed: Feb. 5, 2007

Related U.S. Application Data
(60) Provisional application No. 60/773,656, filed on Feb. 16, 2006.

(30) Foreign Application Priority Data
Feb. 3, 2006 (FR) 06/50392

(51) Int. Cl. A61K 8/49; A61Q 17/04

(52) U.S. Cl. 424/60; 424/59; 424/70.9

(57) ABSTRACT

Photostable sunscreen compositions containing at least one cinnamic acid ester UV-B filter and at least one particular s-triazine compound have improved effectiveness with respect to UV-B radiation.

### CROSS-REFERENCE TO PRIORITY/PROVISIONAL APPLICATIONS

This application claims priority under 35 U.S.C. § 119 of FR 06/50392, filed Feb. 3, 2006, and of U.S. Provisional Application No. 60/773,656, Feb. 16, 2006, each hereby expressly incorporated by reference and each assigned to the assignee hereof.

### CROSS-REFERENCE TO COMPANION APPLICATIONS

Copending application Ser. No. ______ [Attorney Docket No. 1016800-000795] and Ser. No. ______ [Attorney Docket No. 1016800-000797], filed concurrently herewith and each also assigned to the assignee hereof.

### BACKGROUND OF THE INVENTION

1. Technical Field of the Invention

The present invention relates to novel sunscreen compositions, in particular cosmetic sunscreen compositions for topical application containing the combination of a cinnamate ester UV-B filter and an s-triazine having two hindered para-aminobenzalmalonate substituents and one para-aminobenzoate substituent.

The present invention also relates to a process for the photostabilization of at least one cinnamate ester UV-B filter against UV radiation by at least one s-triazine compound having two hindered para-aminobenzalmalonate substituents and one para-aminobenzoate substituent.

This invention also relates to the formulation of such s-triazine compounds into compositions containing, in a cosmetically acceptable vehicle, at least one cinnamate ester UV-B filter for the purpose of improving the efficacy of such compositions against the damaging effects of UV-B rays.

2. Description of Background and/or Related and/or Prior Art

It is known that light radiation of wavelengths ranging from 280 nm to 400 nm enable the browning of the human epidermis, and that rays of wavelength more particularly ranging from 280 to 320 nm, known as UV-B, cause erythemas and skin burns which can impair the development of a natural tan. For these reasons and for aesthetic reasons, there is a constant demand for means of controlling this natural tanning with a view to thus controlling the color of the skin; this UV-B radiation should therefore be filtered out.

It is also known that UV-A rays, of wavelengths ranging from 320 to 400 nm, which cause the browning of the skin, are capable of inducing deterioration therein, in particular in the case of sensitive skin or skin continually exposed to solar radiation. The UV-A rays cause, in particular, a loss of elasticity in the skin and the appearance of wrinkles leading to premature skin aging. They facilitate the triggering of the erythematous reaction or amplify that reaction in certain individuals and may even be the cause of phototoxic or photo-allergic reactions. Thus, for aesthetic and cosmetic reasons such as for example the preservation of the natural elasticity of the skin, more and more individuals with to control the effect of the UV-A rays on their skin. It is thus desirable to also filter out the UV-A radiation.

For the purpose of ensuring protection of the skin and keratinic substrates against the damaging effects of UV radiation, anti-sun/sunscreen compositions containing organic filters active in the UV-A and active in the UV-B, are generally topically applied thereon. The majority of these filters are liposoluble.

In this respect, a particularly interesting family of UV-B filters at present comprises the cinnamate esters and in particular 2-ethylhexyl para-methoxycinnamate, which in fact display a strong intrinsic absorption power. These cinnamate ester compounds, which are per ser now well known as active filters in the UV-B region, are in particular described in FR-A-2,315,908; moreover, 2-ethylhexyl para-methoxycinnamate is currently commercially available under the trademark “PARSOL MCX” by the DSM company.

Unfortunately, it is found that the cinnamate esters are substances which are relatively sensitive to ultraviolet radiation, that is to say, more precisely, that they have an annoying tendency to degrade more or less rapidly under the action of the latter. Thus, this substantial lack of photochemical stability of the cinnamate ester compounds towards the ultraviolet radiation to which they are by their nature destined to be subjected renders it impossible to guarantee constant protection during prolonged exposure to the sun, so that repeated applications at regular and frequent intervals of time must be performed by the user to obtain effective protection of the skin against the UV rays.

### SUMMARY OF THE INVENTION

It has now surprisingly been discovered that a novel family of s-triazine compounds of formula (I) below, having two substituents selected from hindered para-aminobenzalmalonate and para-aminobenzalmalonamide groups and one aminobenzoate or aminobenzamide substituent make it possible to improve the photochemical stability (or photostability) of cinnamate ester UV-B filters substantially and thus to improve the protective action of sunscreen compositions containing this type of UV-B filter in the region of UV-A radiation.

Moreover, these triazine derivatives of formula (I) display good absorbent properties over the entire range of UV-A rays and a substantial contribution in the range of the UV-B and markedly improved photostability and solubility compared to s-triazine compounds grafted with para-aminobenzalmalonates of the prior art, such as those described in EP-0,507,691, in particular the derivatives 2,4-bis-(di-2-ethylhexyl 4′-aminobenzalmalonate)-6-(2-ethylhexyl 4′-aminobenzoate)-s-triazine and 2,4-bis-(di-2-ethylhexyl 4′-aminobenzalmalonate)-6-(2-ethylhexyl 5′-amino salicylate)-s-triazine or in EP-0,841,341 such as, for example, 2,4-bis (diisobutyl 4′-diylaminobenzalmalonate)-6-{[1,3,3,3-tetramethyl-1-[(trimethylsilyl)oxy]disiloxanyl]propyl-3-ylamino}-s-triazine.

Furthermore, these same unique s-triazine compounds of formula (I) are photostable even in the presence of an ester of cinnamic acid.

These discoveries are the basis of the present invention.

Thus, the present invention features novel compositions which comprise, formulated into a physiologically acceptable vehicle, at least one UV filter system comprising:

(a) at least one cinnamate ester UV-B filter and

(b) at least one s-triazine compound of formula (I) below.

The present invention also features a process for improving the chemical stability of at least one cinnamate ester UV-B filter towards UV radiation, which comprises combining with such ester of cinnamic acid at least one s-triazine compound of formula (I) below.

This invention also features formulating an s-triazine derivative of formula (I) below, into a composition containing, in a physiologically acceptable vehicle, at least one cinnamate ester UV-B filter for the purpose of improving the chemical stability of the said cinnamate ester compound towards UV rays.

The present invention also features formulating an s-triazine compound of formula (I) below, in a composition containing, in a cosmetically acceptable vehicle, at least one cinnamate ester UV-B filter for the purpose of improving the efficacy of such composition against UV-B rays.

Other characteristics, aspects and advantages of the invention will become apparent from the detailed description to follow.

In the present description, “system filtering UV radiation” means an agent filtering UV radiation, comprising either a single organic or inorganic compound filtering UV radiation or a mixture of several organic or inorganic compounds filtering UV radiation, for example a mixture containing a UV-A filter and a UV-B filter.

“Cosmetically acceptable” means compatible with the skin and/or integuments thereof, and which displays an agreeable color, odor and touch and does not cause unacceptable discomfort (smarting, tightness, redness) capable of discouraging the consumer from applying that composition.

### DETAILED DESCRIPTION OF BEST MODE AND SPECIFIC/PREFERRED EMBODIMENTS OF THE INVENTION

The cinnamate esters according to the invention are preferably selected from among those having the following formula (A): 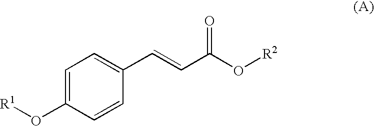
wherein:

the radicals R¹ and R², which may be identical or different, are each a linear or branched C₁-C₂₄ alkyl radical, and more particularly a linear or branched C₁-C₈ alkyl radical, such as methyl, ethyl, propyl, isobutyl, butyl, sec. butyl, isobutyl, pentyl, neopentyl, hexyl and 2-ethylhexyl.

Among the cinnamate ester compounds of formula (A), the following are particularly exemplary:

Ethylhexyl methoxycinnamate or Cinoxate, commercially available under the trademark “PARSOL MCX” from DSM

Isopropyl methoxy cinnamate,

Isoamyl methoxy cinnamate commercially available under the trademark “NEO HELIOPAN E 1000” from SYMRISE,

Diisopropyl methylcinnamate.

The cinnamate ester or esters are present in the compositions according to the invention in amounts which preferably ranging from 0.01% to 20% by weight and more preferably from 0.1% to 10% by weight and still more preferably from 0.1% to 8% by weight relative to the total weight of the composition.

The s-triazine compounds according to the present invention have the following general formula (I): 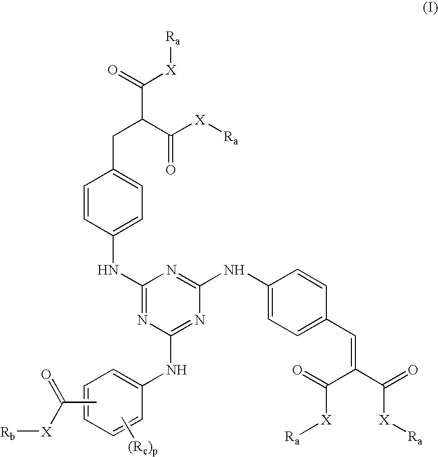
wherein:

the groups X, which may be the same or different, are each —O— or —NR₆—;

the radicals Rₐ, which may be the same or different, are each a radical of formula (II): 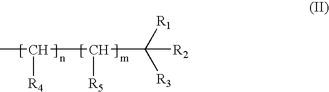
wherein:

the radicals R₁ and R₂, which may be the same or different, are each a linear or branched C₁-C₈ alkyl radical, with the proviso that R₁ and R₂ may together form a C₅-C₈ ring member, optionally substituted with 1, 2 or 3 linear or branched C₁-C₄ alkyl radicals;

the radicals R₃, R₄ and R₅, which may be the same or different, are each a hydrogen atom or a linear or branched C₁-C₄ alkyl radical;

n equals 0 or 1;

m equals 0 or 1;

with the proviso that

(i) when n=1 and R₄ is hydrogen, then m equals 0 and R₃ is other than hydrogen;

(ii) when R₁ and R₂ form a C₅-C₈ ring member, then the sum n+m is other than 2;

R₆ is hydrogen or a C₁-C₈ alkyl radical;

R_{b} is a linear or branched, and optionally unsaturated, C₁-C₂₀ alkyl radical, a C₅-C₁₂ cycloalkyl radical, optionally substituted with 1 to 3 linear or branched C₁-C₄ alkyl radicals, the radical —(CH₂CHR₇—O)_{q}R₈ or the radical —CH₂—CH(OH)—CH₂—O—R₈;

R₇ is hydrogen or methyl;

R₈ is hydrogen or a C₁-C₈ alkyl radical;

q=1-20;

the COXR_{b} group may be in the ortho, meta or para position to the amino group;

R_{c} is a linear or branched, saturated or unsaturated C₁-C₂₀ alkyl radical, the OH group or a linear or branched C₁-C₂₀ alkoxy radical; and

p equals 0, 1 or 2.

In the above formula (I), the alkyl radicals can, in particular, be selected from among the methyl, ethyl, n-propyl, isopropyl, n-butyl, isobutyl, tert-butyl, n-amyl, isoamyl, neopentyl, n-hexyl, n-heptyl, n-octyl, 2-ethylhexyl and tert-octyl radicals. The particularly preferred alkyl radical is the methyl radical.

The cycloalkyl radicals can, in particular, be selected from among the cyclopentyl, cyclohexyl and cycloheptyl radicals. The particularly preferred cycloalkyl radical is the cyclohexyl radical. These radicals may be substituted with C₁-C₄ alkyl groups preferably selected from among methyl, isopropyl and tert-butyl.

Among the preferred compounds of formula (I), those for which the following two conditions are both satisfied are exemplary:

(a) n=m=0, and

(b) R₁, R₂ and R₃ are each a C₁-C₄ alkyl radical and more particularly methyl or else R₃ is hydrogen and R₁ and R₂ together form a C₅-C₈ ring member, optionally substituted with 1 or two alkyl radicals and more particularly cyclohexyl.

Also among the preferred compounds of formula (I), those for which the following two conditions are both satisfied too are exemplary:

(a) n=1 and R₄ is an alkyl radical in particular methyl or m=1 and R₅ is an alkyl in particular methyl and

(b) R₁ and R₂ are each a C₁-C₄ alkyl and more particularly methyl.

Among the more particularly preferred compounds of formula (I), those selected from among the following compounds of formulae (1) to (10) are representative:

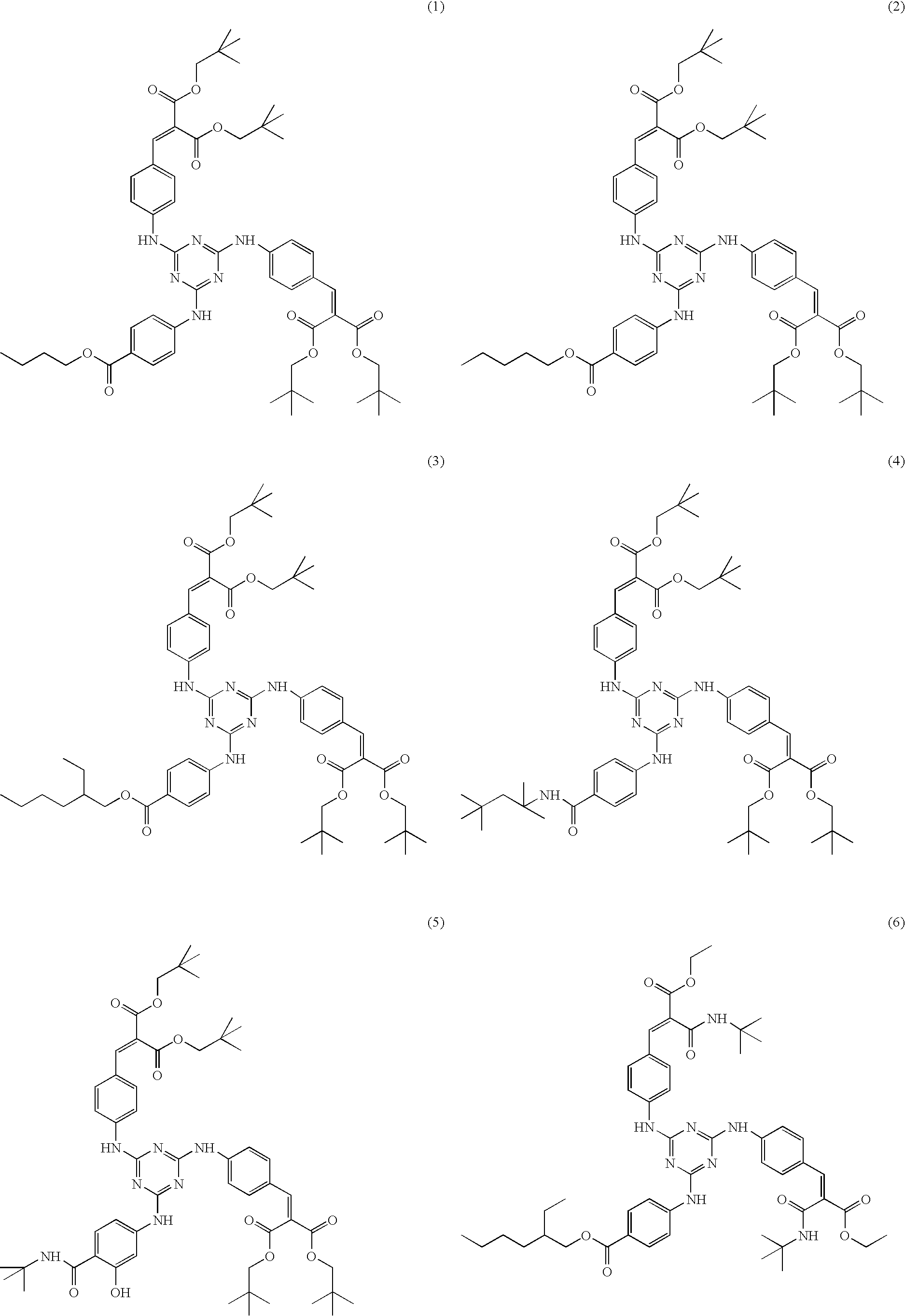

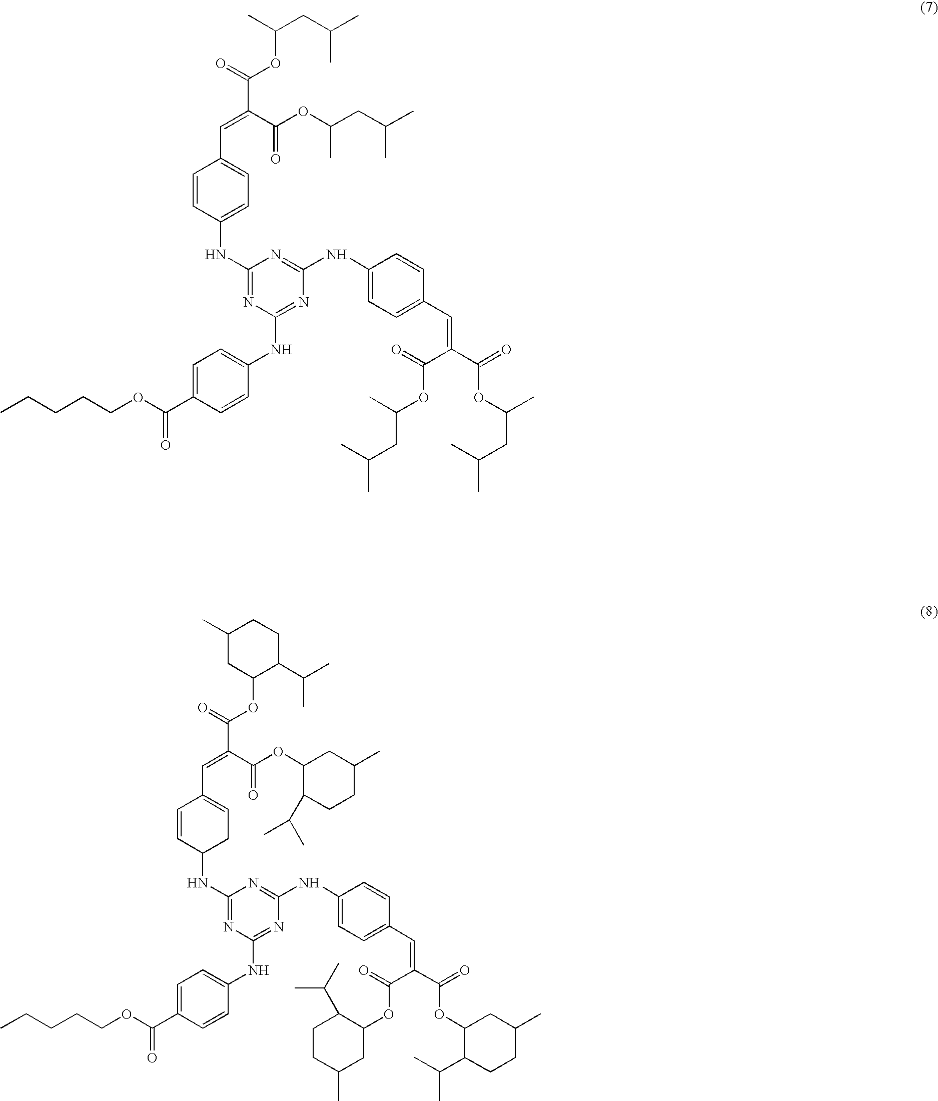

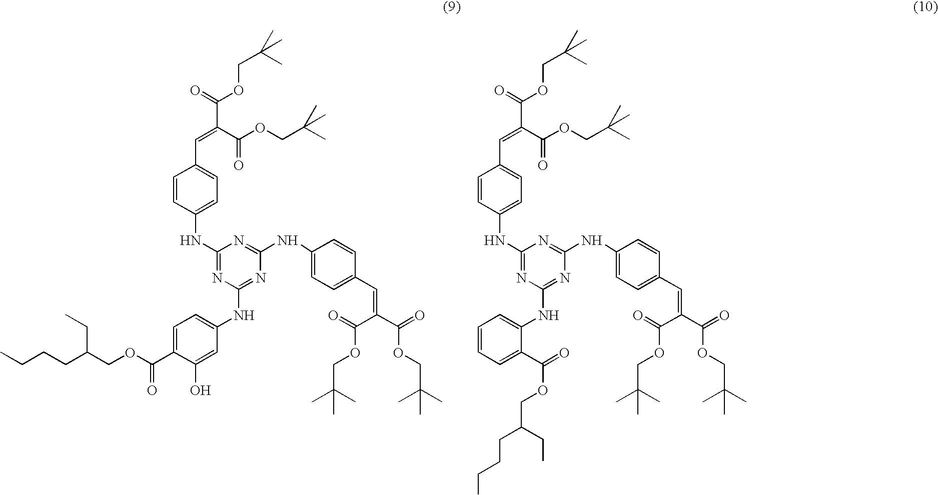

Among these compounds, 2,4-bis-(di-neopentyl 4′-aminobenzalmalonate)-6-(butyl 4″-aminobenzoate)-s-triazine of formula (1) is more particularly preferred.

The compounds of formula (I) can be prepared according to the following scheme (A): 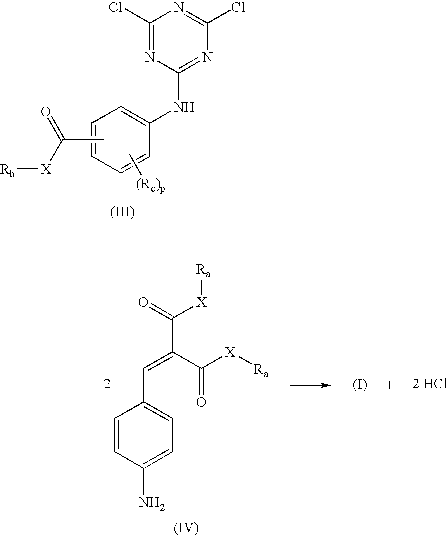
wherein Rₐ, X, R_{b}, R^{c} and p have the definitions in the above formula (1).

The above reactions can be performed, optionally in the presence of a solvent (for example: toluene, xylene or 1,2-dichloroethane), at a temperature ranging from 0° C. to 250° C., more particularly from 5° C. to 150° C. They can also be carried out with microwaves in the presence or absence of a solvent (for example: toluene, xylene or 1,2-dichloroethane) or in the presence or absence of 10% of graphite, at a temperature of 50° C. to 150° C., and 50-150 watts power for a period of 10 to 30 minutes.

The compounds of formula (III) can be prepared according to known methods described, for example, in EP-0,507,691.

The compounds of formula (1) are generally present in the compositions of the invention in proportions ranging from 0.01% to 20% by weight, preferably from 0.1% to 10% by weight, relative to the total weight of the composition.

The compositions according to the invention can also contain other complementary organic or inorganic UV filters active in the UV-A and/or UV-B regions, which are water-soluble or liposoluble or else insoluble in the cosmetic solvents commonly employed.

The complementary organic filters are in particular selected from among the anthranilates, salicylate derivatives, camphor derivatives, triazine derivatives other than those of the invention, such as those described in U.S. Pat. No. 4,367,390, EP-863,145, EP-517,104, EP-570,838, EP-796,851, EP-775,698, EP-878,469, EP-933,376, EP-507,691, EP-507,692, EP-790,243 and EP-944,624, benzophenone derivatives, β,β-diphenylacrylate derivatives, benzotriazole derivatives, benzalmalonate derivatives other than those of the invention, benzimidazole derivatives, imidazolines, bis-benzoazolyl derivatives as described in EP-669,323 and U.S. Pat. No. 2,463,264, p-aminobenzoic acid (PABA) derivatives, methylene bis-(hydroxyphenyl benzotriazole) derivatives as described in U.S. Pat. Nos. 5,237,071 and 5,166,355, GB-2,303,549, DE-197,26,184 and EP-893,119, filter polymers and filter silicones such as those described in particular in WO-93/04665, dimers derived from α-alkylstyrene such as those described in DE-198,55,649, 4,4-diarylbutadienes as described in EP-0,967,200, DE-197,46,654, DE-197,55,649, EP-A-1,008,586, EP-1,133,980 and EP-133,981 and mixtures thereof.

As examples of complementary organic filters, those indicated below under their INCI name are representative:

Para-Aminobenzoic Acid Derivatives:

PABA,

Ethyl PABA,

Ethyl dihydroxypropyl PABA,

Ethylhexyl dimethyl PABA marketed in particular under the trademark “ESCALOL 507” by ISP,

Glyceryl PABA,

PEG-25 PABA marketed under the trademark “UVINUL P25” by BASF.

Salicylate Derivatives:

Homosalate marketed under the trademark “Eusolex HMS” by Rona/EM Industries,

Ethylhexyl salicylate marketed under the trademark “NEO HELIOPAN OS” by HAARMANN and REIMER,

Dipropylene glycol salicylate marketed under the trademark “DIPSAL” by SCHER,

TEA salicylate, marketed under the trademark “NEO HELIOPAN TS” by HAARMANN and REIMER.

β,β′-diphenylacrylate Derivatives:

Octocrylene marketed in particular under the trademark “UVINUL N539” by BASF,

Etocrylene, marketed in particular under the trademark “UVINUL N35” by BASF.

Benzophenone Derivatives:

Benzophenone-1 marketed under the trademark “UVINUL 400” by BASF,

Benzophenone-2 marketed under the trademark “UVINUL D50” by BASF

Benzophenone-3 or Oxybenzone, marketed under the trademark “UVINUL M40” by BASF,

Benzophenone-4 marketed under the trademark “UVINUL MS40” by BASF, Benzophenone-5

Benzophenone-6 marketed under the trademark “Helisorb 11” by Norquay

Benzophenone-8 marketed under the trademark “Spectra-Sorb UV-24” by American Cyanamid

Benzophenone-9 marketed under the trademark “UVINUL DS-49” by BASF, Benzophenone-12

Diethylamino hydroxybenzoyl hexyl benzoate marketed under the trademark “UVINUL A PLUS” by BASF.

Benzylidene Camphor Derivatives:

3-benzylidene camphor manufactured under the trademark “MEXORYL SD” by CHIMEX,

4-methylbenzylidene camphor marketed under the trademark “EUSOLEX 6300” by MERCK,

Benzylidene camphor sulfonic acid manufactured under the trademark “MEXORYL SL” by CHIMEX,

Camphor Benzalkonium methosulfate manufactured under the trademark “MEXORYL SO” by CHIMEX,

Terephthalylidene dicamphor sulfonic acid manufactured under the trademark “MEXORYL SX” by CHIMEX,

Polyacrylamidomethyl benzylidene camphor manufactured under the trademark “MEXORYL SW” by CHIMEX.

Phenyl Benzimidazole Derivatives:

Phenylbenzimidazole sulfonic acid marketed in particular under the trademark “EUSOLEX 232” by MERCK,

Disodium phenyl dibenzimidazole tetra-sulfonate marketed under the trademark “NEO HELIOPAN AP” by HAARMANN and REIMER.

Triazine Derivatives:

Bis-ethylhexyloxyphenol methoxyphenyl triazine marketed under the trademark “TINOSORB S” by CIBA GEIGY,

Ethylhexyl triazone marketed in particular under the trademark “UVINUL T150” by BASF,

Diethylhexyl butamido triazone marketed under the trademark “UVASORB HEB” by SIGMA 3V.

Phenyl Benzotriazole Derivatives:

Drometrizole trisiloxane marketed under the trademark “Silatrizole” by RHODIA CHIMIE,

Methylene bis-benzotriazolyl tetramethylbutylphenol, marketed in solid form under the trademark “MIXXIM BB/100” by FAIRMOUNT CHEMICAL or in micronized form in aqueous dispersion under the trademark “TINOSORB M” by CIBA SPECIALTY CHEMICALS.

Anthranilate Derivatives:

Menthyl anthranilate marketed under the trademark “NEO HELIOPAN MA” by HAARMANN and REIMER.

Imidazoline Derivatives:

Ethylhexyl dimethoxybenzylidene dioxoimidazoline propionate.

Benzalmalonate Derivatives:

Benzalmalonate group-containing polyorganosiloxanes such as Polysilicone-15 marketed under the trademark “PARSOL SLX” by HOFFMANN LA ROCHE.

4,4-diarylbutadiene Derivatives:

-1,1-dicarboxy (2,2′-dimethyl-propyl)-4,4-diphenylbutadiene and mixtures thereof.

The preferred complementary organic UV filters are selected from among:

Ethylhexyl salicylate,

Homosalate,

Octocrylene,

Phenylbenzimidazole sulfonic acid,
Disodium phenyl dibenzimidazole tetra-sulfonate,
Benzophenone-3,

Benzophenone-4,

Benzophenone-5,

n-hexyl 2-(4-diethylamino-2-hydroxybenzoyl)-benzoate,

4-methylbenzylidene camphor,

Terephthalylidene dicamphor sulfonic acid,

Anisotriazine,

Ethylhexyl triazone,

Diethylhexyl butamido triazone,

Methylene bis-benzotriazolyl tetramethylbutylphenol,

Drometrizole trisiloxane,

Polysilicone-15,

1,1-dicarboxy (2,2′-dimethyl-propyl)-4,4-diphenylbutadiene and mixtures thereof.

The complementary inorganic photoprotective agents are selected from among the pigments and still more preferably from the pigments having a mean size of the primary particles: generally ranging from 5 nm to 100 nm, preferably from 10 nm to 50 nm of treated or untreated metal oxides such as, for example, pigments oxides of titanium (amorphous or crystalline in rutile and/or anatase form), iron, zinc, zirconium or cerium.

Treated pigments are pigments which have undergone one or several surface treatments of a chemical, electronic, mechanochemical and/or mechanical nature with derivatives as described, for example, in Cosmetics & Toiletries, February 1990, Vol. 105, p 53-64, such as amino acids, beeswax, fatty acids, fatty alcohols, anionic surfactants, lecithins, sodium, potassium, zinc, iron or aluminum salts of fatty acids, metal alkoxides (of titanium or aluminum), polyethylene, silicones, proteins (collagen, elastin), alkanolamines, silicon oxides, metal oxides, sodium hexametaphosphate, alumina or glycerine.

More particularly, the treated pigments can be oxides of titanium treated with:

silica and alumina, such as the products “Microtitanium Dioxide MT 500 SA” and “Microtitanium Dioxide MT 100 SA” from the TAYCA company, and the products “Tioveil Fin”, “Tioveil OP”, “Tioveil MOTG” and “Tioveil IPM” from the TIOXIDE company,

alumina and aluminum stearate, such as the product “Microtitanium Dioxide MT 100 T” from the TAYCA company,

alumina and aluminum laurate, such as the product “Microtitanium Dioxide MT 100 S” from the TAYCA company,

iron oxides and iron stearate, such as the product “Microtitanium Dioxide MT 100 F” from the TAYCA company,

silica, alumina and silicone, such as the products “Microtitanium Dioxide MT 100 SAS”, “Microtitanium Dioxide MT 600 SAS” and “Microtitanium Dioxide MT 500 SAS” from the TAYCA company,

sodium hexametaphosphate, such as the product “Microtitanium Dioxide MT 150 W” from the TAYCA company,

octyltrimethoxysilane, such as the product “T-805” from the DEGUSSA company,

alumina and stearic acid, such as the product “UVT-M160” from the KEMIRA company,

alumina and glycerine, such as the product “UVT-M212” from the KEMIRA company, and

alumina and silicone, such as the product “UVT-M262” from the KEMIRA company.

Other pigments of titanium oxide treated with a silicone are preferably TiO₂ treated with octyl trimethyl silane and wherein the mean size of the elementary particles ranges from 25 to 40 nm, such as that marketed under the trademark “T 805” by the DEGUSSA SILICES company, TiO2 treated with a polydimethylsiloxane and wherein the mean size of the elementary particles is 21 nm, such as that marketed under the trademark “70250 Cardre UF TiO2SI3” by the CARDRE company, anatase/rutile TiO2 treated with a polydimethylhydrogenosiloxane and wherein the mean size of the elementary particles is 25 nm, such as that marketed under the trademark “MICRO TITANIUM DIOXYDE USP GRADE HYDROPHOBIC” by the COLOR TECHNIQUES company.

Uncoated titanium oxide pigments are, for example, marketed by the TAYCA company under the trademarks “MICROTITANIUM DIOXIDE MT 500 B” or “MICROTITANIUM DIOXIDE MT600 B”, by the DEGUSSA company under the trademark “P 25”, by the WACKER company under the trademark “Transparent titanium oxide PW”, by the MIYOSHI KASEI company under the trademark “UFTR”, by the TOMEN company under the trademark “ITS” and by the TIOXIDE company under the trademark “TIOVEIL AQ”.

Uncoated zinc oxide pigments are, for example:

those marketed under the trademark “Z-cote” by the Sunsmart company;

those marketed under the trademark “Nanox” by the Elementis company, and

those marketed under the trademark “Nanogard WCD 2025” by the Nanophase Technologies company.

Coated zinc oxide pigments are, for example:

those marketed under the trademark “Oxide zinc CS-5” by the Toshibi company (ZnO coated with polymethylhydrogensiloxane),

those marketed under the trademark “Nanogard Zinc Oxide FN” by the Nanophase Technologies company (as 40% dispersion in Finsolv TN, benzoate of C₁₂-C₁₅ alcohols),

those marketed under the trademarks “DAITOPERSION ZN-30” and “DAITOPERSION Zn-50” by the Daito company (dispersions in ethoxylated cyclopolymethylsiloxane/polydimethylsiloxane, containing 30% or 50% of zinc oxides coated with silica and polymethylhydrogensiloxane),

those marketed under the trademark “NFD Ultrafine ZnO” by the Daikin company (ZnO coated with perfluoroalkyl phosphate and perfluoroalkylethyl-based copolymer as dispersion in cyclopentasiloxane),

those marketed under the trademark “SPD-Z1” by the Shin-Etsu company (ZnO coated with silicone grafted acrylic polymer, dispersed in cyclodimethylsiloxane),

those marketed under the trademark “Escalol Z100” by the ISP company (ZnO treated [with] alumina and dispersed in the mixture ethylhexyl methoxycinnamate/PVP-hexadecene copolymer/methicone),

those marketed under the trademark “Fuji ZnO-SMS-10” by the Fuji Pigment company (ZnO coated [with] silica and polymethylsilsesquioxane),

those marketed under the trademark “Nanox Gel TN” by the Elementis company (55% ZnO dispersed in benzoate of C₁₂-C₁₅ alcohols with hydroxystearic acid polycondensation product).

Uncoated cerium oxide pigments are marketed under the trademark “COLLOIDAL CERIUM OXIDE” by the RHONE POULENC company. Uncoated iron oxide nanopigments are, for example, marketed by the ARNAUD company under the trademarks “NANOGARD WCD 2002 (FE 45B)”, “NANOGARD IRON FE 45 BL AQ”, “NANOGARD FE 45R AQ and “NANOGARD WCD 2006 (FE 45R)”, or by the MITSUBISHI company under the trademark “TY-220”.

Coated iron oxide pigments are, for example, marketed by the ARNAUD company under the trademarks “NANOGARD WCD 2008 (FE 45B FN)”, “NANOGARD WCD 2009 (FE 45B 556)”, “NANOGARD FE 45 BL 345” and “NANOGARD FE 45 BL”, or by the BASF company under the trademark “TRANSPARENT IRON OXIDE”.

Mixtures of metal oxides are also exemplary, in particular mixtures of titanium dioxide and cerium dioxide, including the equal weight mixture of titanium dioxide and cerium dioxide coated with silica, marketed by the IKEDA company under the trademark “SUNVEIL A”, and the mixture of titanium dioxide and zinc dioxide coated with alumina, silica and silicone, such as the product “M 261” marketed by the KEMIRA company or coated with alumina, silica and glycerine, such as the product “M 211” marketed by the KEMIRA company.

The pigments can be introduced into the compositions according to the invention as such or in the form of a pigment paste, that is to say, mixed with a dispersant, as described for example in GB-A-2,206,339.

The additional photoprotective agents are generally present in the compositions according to the invention in proportions ranging from 0.01% to 20% by weight relative to the total weight of the composition, and preferably ranging from 0.1% to 10% by weight relative to the total weight of the composition.

The compositions according to the invention may also contain tanning agents and/or artificial skin browning agents (self-tanning agents), and more particularly dihydroxyacetone (DHA). They are preferably present in quantities ranging from 0.1% to 10% by weight relative to the total weight of the composition.

The compositions according to the present invention can further contain standard cosmetic additives, selected in particular from among fatty substances, organic solvents, ionic or nonionic, hydrophilic or lipophilic thickeners, softeners, humectants, opacifiers, stabilizers, emollients, silicones, anti-foaming agents, perfumes, preservatives, anionic, cationic, nonionic, zwitterionic or amphoteric surfactants, active agents, fillers, polymers, propellants, basifying or acidifying agents or any other ingredient normally used in the cosmetic and/or dermatological fields.

The fatty substances can be an oil or a wax or mixtures thereof. An oil means a substance which is liquid at ambient temperature. A wax means a substance which is solid or essentially solid at ambient temperature, and whose melting point is generally greater than 35° C.

As oils, exemplary are mineral oils (paraffin), vegetable oils (sweet almond, macadamia, blackcurrant seed, or jojoba oils), synthetic oils such as perhydrosqualene, fatty alcohols, acids or esters (such as the benzoate of C₁₂-C₁₅ alcohols marketed under the trademark “Finsolv TN” by the WITCO company, octyl palmitate, isopropyl lanolate, and triglycerides, including those of capric/caprylic acids), ethoxylated or propoxylated fatty esters and ethers, silicone oils (cyclomethicone, polydimethysiloxanes or PDMS) or fluorinated oils and polyalkylenes.

As waxy substances, paraffin, carnauba wax, beeswax and hydrogenated castor oil are exemplary.

Among the organic solvents, alcohols and lower polyols are exemplary. The latter may be selected from the glycols and glycol ethers such as ethylene glycol, propylene glycol, butylene glycol, dipropylene glycol or diethylene glycol.

As hydrophilic thickeners, exemplary are carboxyvinyl polymers such as the carbopols (carbomers) and the Pemulen (acrylate/C10-C30 alkylacrylate copolymer), polyacrylamides such as for example the crosslinked copolymers marketed under the trademarks Sepigel 305 (C.T.F.A. name: polyacrylamide/C13-14 isoparaffin/Laureth 7) or Simulgel 600 (C.T.F.A. name: acrylamide/sodium acryloyldimethyltaurate copolymer/isohexadecane/polysorbate 80) by the Seppic company, polymers and copolymers of 2-acrylamido 2-methyl-propane sulfonic acid, which may be crosslinked and/or neutralized, such as the poly(2-acrylamido 2-methylpropane sulfonic acid) marketed by the Hoechst company under the trademark “Hostacerin AMPS” (CTFA name: ammonium polyacryidimethyltauramide), cellulose derivatives such as hydroxyethylcellulose, polysaccharides and in particular gums such as xanthan gum, and mixtures thereof.

As lipophilic thickeners, exemplary are modified clays such as hectorite and derivatives thereof, such as the products marketed under the trademark Bentone.

Among the active agents, the following are exemplary:

anti-pollution agents and/or anti-radical agents;

depigmentation agents and/or propigmentation agents,

anti-glycation agents,

NO-synthase inhibitors,

agents stimulating the synthesis of dermal or epidermal macromolecules and/or hindering their degradation,

agents stimulating the proliferation of fibroblasts,

agents stimulating the proliferation of keratinocytes,

myorelaxant agents,

tightening agents,

desquamating agents,

hydrating agents,

anti-inflammatory agents,

agents acting on the energy metabolism of cells,

insect repellent agents,

antagonists of P substances or of CRGP.

Of course, one skilled in this art will take care to select any complementary derivative or derivatives mentioned above and/or the quantities thereof in such a manner that the advantageous properties intrinsically associated with the compositions according to the invention are not, or essentially not, impaired by the addition or additions envisaged.

The compositions according to the invention may be formulated by techniques well known to this are, in particular those suited for the preparation of emulsions of the oil-in-water or water-in-oil type. They may in particular be in the form of a simple or complex emulsion (O/W, W/O, O/W/O or W/O/W) such as a cream, a milk, or in the form of a gel or a cream gel, in the form of a lotion, powder or solid rod and may be packaged as an aerosol, and be in the form of a foam or spray.

Preferably, the compositions according to the invention are in the form of an oil-in-water or water-in-oil emulsion.

The emulsions generally contain at least one emulsifier selected from among the amphoteric, anionic, cationic or nonionic emulsifiers, either alone or as a mixture. The emulsifiers are appropriately selected depending on the emulsion to be obtained (W/O or O/W).

As emulsifying surfactants useful for the preparation of W/O emulsions, exemplary are the alkyl esters or ethers of sorbitan, of glycerol or of sugars, silicone surfactants such as the dimethicone copolyols such as the mixture of cyclomethicone and dimethicone copolyol, marketed under the trademark “DC 5225 C” by the Dow Corning company, and alkyl-dimethicone copolyols such as the Laurylmethicone copolyol marketed under the trademark “Dow Corning 5200 Formulation Aid” by the Dow Corning company, Cetyl dimethicone copolyol such as the product marketed under the trademark Abil EM 90R by the Goldschmidt company and the mixture of cetyl dimethicone copolyol, polyglycerol (4 moles) isostearate and hexyl laurate marketed under the trademark ABIL WE 09 by the Goldschmidt company. To these may also be added one or several co-emulsifiers, which may advantageously be selected from the group comprising the polyol alkyl esters. As polyol alkyl esters, exemplary are the esters of glycerol and/or of sorbitan and for example polyglycerol isostearate, such as the product marketed under the trademark Isolan GI 34 by the Goldschmidt company, sorbitan isostearate, such as the product marketed under the trademark Arlacel 987 by the ICI company, sorbitan isostearate and glycerol, such as the product marketed under the trademark Arlacel 986 by the ICI company, and mixtures thereof.

For O/W emulsions, exemplary emulsifiers are nonionic emulsifiers such as alkoxylated (more particularly polyethoxylated) esters of fatty acids and glycerol, alkoxylated esters of fatty acids and sorbitan, alkoxylated (ethoxylated and/or propoxylated) esters of fatty acids, alkoxylated (ethoxylated and/or propoxylated) ethers of fatty alcohols, esters of sugars such as sucrose stearate, fatty alcohol and sugar ethers, in particular alkylpolyglucosides (APG) such as the decylglucoside and laurylglucoside marketed for example by the Henkel company under the trademarks Plantaren 2000 and Plantaren 1200, cetostearylglucoside optionally mixed with cetostearyl alcohol, marketed for example under the trademark Montanov 68 by the Seppic company, under the trademark Tegocare CG90 by the Goldschmidt company and under the trademark Emulgade KE3302 by the Henkel company, and also arachidyl glucoside, for example in the form of the mixture of arachidic and behenic alcohols and arachidylglucoside marketed under the trademark Montanov 202 by the Seppic company. According to one particular embodiment of the invention, the mixture of the alkylpolyglucoside as defined above with the corresponding fatty alcohol may be in the form of a self-emulsifying composition, as described, for example, in WO-A-92/06778.

In the case of an emulsion, the aqueous phase of the latter may contain a nonionic vesicle dispersion prepared by known processes (Bangham, Standish and Watkins, J. Mol. Biol., 13, 238 (1965), FR-2,315,991 and FR-2,416,008).

The compositions according to the invention are administered in a large number of treatments, whether regime or regimen, in particular cosmetic, of the skin, the lips and the hair, including the scalp, in particular for the protection and/or care of the skin, the lips and/or the hair, and/or for making up the skin and/or the lips.

The present invention also features the use of the compositions according to the invention as defined above for the manufacture of products for the cosmetic treatment of the skin, the lips, the nails, the hair, the eyelashes, eyebrows and/or the scalp, in particular care products and makeup products.

The cosmetic compositions according to the invention are useful, for example, as a face and/or body care and/or sun protection product of liquid to semi-liquid consistency, such as milks, more or less smooth creams, gel-creams and pastes. They may optionally be packaged as aerosols and be in the form of a foam or spray.

The compositions according to the invention in the form of vaporizable liquid lotions are applied onto the skin or the hair in the form of fine particles by means of pressurization devices. The devices according to the invention are well known to those skilled in this art and include non-aerosol pumps or atomizers, aerosol containers containing a propellant and also aerosol pumps employing compressed air as the propellant. The latter are described in U.S. Pat. Nos. 4,077,441 and 4,850,517.

The compositions packaged as aerosols according to the invention generally contain conventional propellant agents such as for example hydrofluorinated derivatives, dichlorodifluoromethane, difluoroethane, dimethyl ether, isobutane, n-butane, propane, and trichlorofluoromethane. They are preferably present in quantities ranging from 15% to 50% by weight relative to the total weight of the composition.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Synthesis of 2,4-bis-(di neopentyl 4′-aminobenzalmalonate)-6-(butyl 4″-aminobenzoate)-s-triazine of Formula (1)

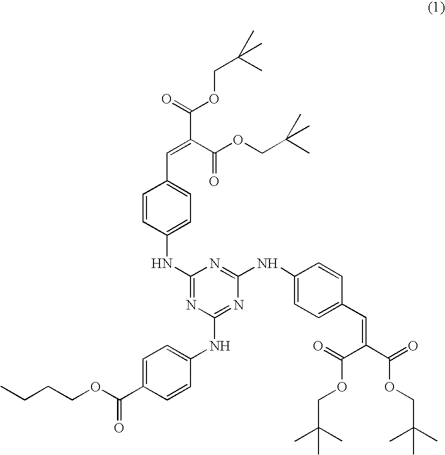

First Stage: Preparation of 2,4-dichloro-6-(butyl 4′-aminobenzoate)-s-triazine:

In a reactor, cyanuric chloride (20.7 g, 0.112 mole) is dissolved in 250 ml of acetone at 0° C.-5° C. A solution of butyl para-aminobenzoate (21.7 g, 0.112 mole) dissolved in 70 ml of acetone is added to this drop by drop at 0° C.-5° C. in 1 hour. Next, sodium bicarbonate (9.4 g, 0.112 mole) dissolved in 70 ml of water is added to this. The heterogeneous mixture is left for 2 hours at a temperature of 0° C.-5° C. The precipitate formed is filtered off, then washed with water and acetone. After drying under vacuum, 37.2 g (yield 97%) of 2,4-dichloro-6-(butyl 4′-aminobenzoate)-s-triazine is obtained in the form of a white powder:

UV (Ethanol/DMSO): λmax=298 nm, E1%=940, and used as such in the following stage.

Second Stage: Preparation of the Compound of Example 1:

With nitrogen aeration, a mixture of the previous product (7.41 g, 0.0213 mole) and dineopentyl para-aminobenzalmalonate (14.66 g, 0.0422 mole) suspended in 60 ml of toluene is heated under reflux for 7 hours 30 minutes. It is cooled and dichloromethane is added. The organic phase is washed with a saturated solution of sodium bicarbonate then with water. The organic phase is dried then concentrated under reduced pressure. The orange oil obtained (17.8 g) is subjected to separation on a silica column (eluent: heptane/EtOAc 85:15). Clean fractions are recovered in the form of pale yellow flakes of the compound of example 1 (8.72 g, yield 43%):

UV (Ethanol): λ=370 nm, E1%=623; λmax=347 nm, E1%=847; λ=300 nm, E1%=432.

### Example 2

### Synthesis of 2,4-bis-(dineopentyl 4′-aminobenzalmalonate)-6-(amyl 4″-aminobenzoate)-s-triazine of Formula (2)

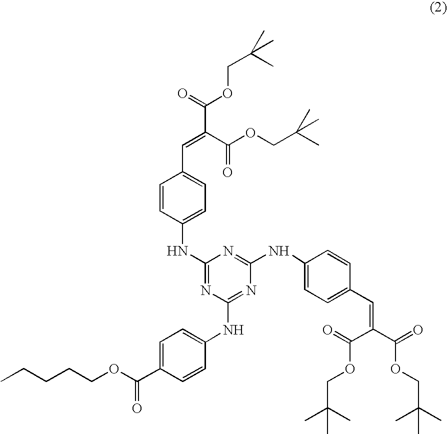

First Stage: Preparation of 2,4-dichloro-6-(amyl 4′-aminobenzoate)-s-triazine:

In a reactor, cyanuric chloride (14.7 g, 0.0796 mole) is dissolved in 200 ml of dioxane at 10° C. A solution of amyl para-aminobenzoate (16.5 g, 0.0796 mole) dissolved in 60 ml of dioxane and a solution of potassium carbonate (5.5 g, 0.0398 mole) dissolved in 30 ml of water are added simultaneously to this drop by drop at 10° C. in 1 hour. The heterogeneous mixture is left for 2 hours at a temperature of 10° C. About 300 ml of water are added and the precipitate formed is filtered off, then washed with water. After drying under vacuum, 26.4 g (yield 93%) of 2,4-dichloro-6-(amyl 4′-aminobenzoate)-s-triazine are obtained and used as such in the following stage.

Second Stage: Preparation of the Compound of Example 2:

An intimate mixture of the previous product (0.103 g, 0.29×10⁻³ mole), dineopentyl para-aminobenzalmalonate (0.2 g, 0.58×10⁻³ mole) and sodium bicarbonate (0.049 g, 0.58×10⁻³ mole) is kept in a CEM Discover microwave reactor for 10 minutes at a temperature of 150° C. and 150 watts power. The amorphous solid formed is extracted with dichloromethane. The organic phase is washed 3 times with water and dried then concentrated under reduced pressure. The yellow oil obtained is subjected to separation on a silica column (eluent: heptane/EtOAc 80:20). Clean fractions are recovered in the form of a pale yellow paste of the compound of example 2 (36 mg, yield 15%):

UV (Ethanol): λ=375 nm, E1%=610; λmax=347 nm, E1%=834; λ=300 nm, E1%=425.

### Example 3

### Synthesis of 2,4-bis-(dineopentyl 4′-aminobenzalmalonate)-6-(2-ethylhexyl 4″-aminobenzoate)-s-triazine of Formula (3)

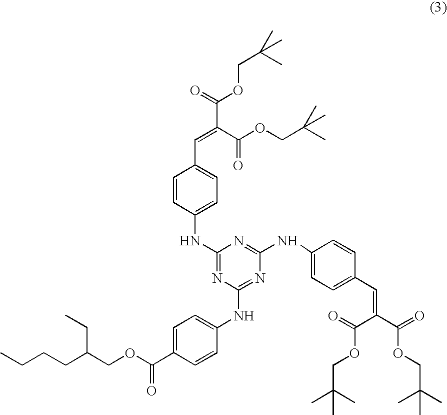

First Stage: Preparation of 2,4-dichloro-6-(2-ethylhexyl 4′-aminobenzoate)-s-triazine:

In a reactor, cyanuric chloride (18.4 g, 0.1 mole) is dissolved in 150 ml of acetone at 0° C.-5° C. Sodium bicarbonate (10.6 g, 0.1 mole) is added, then a solution of 2-ethylhexyl para-aminobenzoate (24.9 g, 0.1 mole) dissolved in 150 ml of acetone is added drop by drop at a temperature below 10° C. in 10 minutes. Next the heterogeneous mixture is left for 3 hours at laboratory temperature. 500 ml of water are poured in. The precipitate formed is filtered off, then washed with water. After drying under vacuum, 38 g of an off-white solid are obtained. After recrystallization of this solid from 1,2-dichloroethane, 25.2 g (yield 63%) of 2,4-dichloro-6-(2-ethylhexyl 4′-aminobenzoate)-s-triazine are obtained in the form of a white powder:

UV (Ethanol/DMSO): λmax=291 nm, E1%=732, and used as such in the following stage.

Second Stage: Preparation of the Compound of Example 3:

With nitrogen aeration, a mixture of the previous product (1.14 g, 2.87×10⁻³ mole) and dineopentyl para-aminobenzalmalonate (2.2 g, 6.33×10⁻³ mole) suspended in 35 ml of toluene is heated under reflux for 10 hours 30 minutes. This is cooled and dichloromethane is added. The organic phase is washed with a saturated solution of sodium bicarbonate then with water. The organic phase is dried then concentrated under reduced pressure. The orange oil obtained (2.6 g) is subjected to separation on a silica column (eluent: heptane/EtOAc 85:15). Clean fractions are recovered in the form of pale yellow flakes of the compound of example 3 (1.17 g, Yield 40%):

UV (Ethanol): λ=370 nm, E1%=575; λmax=342 nm, E1%=880; λ=300 nm, E1%=448.

### Example 4

### Synthesis of 2,4-bis-(dineopentyl 4′-aminobenzalmalonate)-6-(tert-octyl 4″-aminobenzamide)-s-triazine of Formula (4)

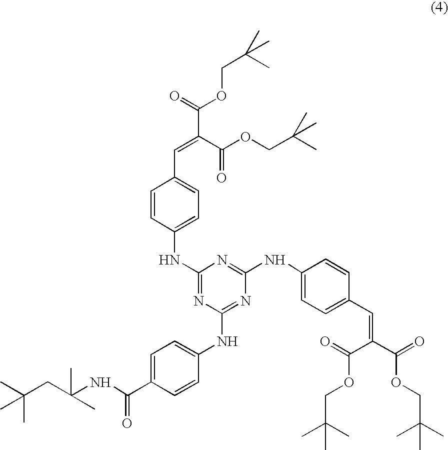

First Stage: Preparation of 4-nitro-N-(tert-octyl)benzamide:

Tert-octyl amine (51.7 g, 0.4 mole) and triethylamine (61.2 ml, 0.44 mole) in 260 ml of dichloroethane are introduced into a reactor. This is heated to 70° C., then 4-nitro-benzoyl chloride (77.9 g, 0.42 mole) is added in small portions in 50 minutes. This is heated under reflux for 4 hours. The mixture is poured onto iced water, extracted with dichloromethane, dried and the solvent evaporated. The beige precipitate obtained is recrystallized from a mixture of isopropyl ether and ethanol (ratio 10:1). After drying under vacuum, 84.6 g (yield 76%) of the 4-nitro-N-(tert-octyl)benzamide are obtained in the form of an off-white powder and used as such in the following stage.

Second Stage: Preparation of 4-amino-N-(tert-octyl)benzamide:

In a 500 ml hydrogenator, 4-nitro-N-(tert-octyl)benzamide (30 g, 0.108 mole) dissolved in 200 ml of ethyl acetate is hydrogenated in the presence of 4.8 g of 10% palladium on carbon with 50% water content as catalyst (hydrogen pressure: 8-10 bars) at a temperature of 70-75° C. for 1 hour and 15 minutes. After filtration, concentration of the solvent and drying under vacuum, 20.4 g (yield: 76%) of 4-amino-N-(tert-octyl)benzamide are obtained in the form of a pale yellow powder and used as such in the following stage.

Third Stage: Preparation of N-(tert-octyl)-4-[(4,6-dichloro-1,3,5-triazin-2-yl)amino]benzamide:

In a reactor, cyanuric chloride (3.7 g, 0.0201 mole) is dissolved in 70 ml of dioxane at 10° C. A solution of the product from the previous stage (5 g, 0.0201 mole) dissolved in 100 ml of dioxane and a solution of potassium carbonate (1.4 g, 0.03 mole) dissolved in 20 ml of water are added simultaneously to this drop by drop at 10° C. in 1 hour. The heterogeneous mixture is left for 2 hours at a temperature of 10° C. About 300 ml of water are added and the precipitate formed is filtered off, then washed with water. After drying under vacuum, 7.4 g (yield 93%) of N-(tert-octyl)-4-[(4,6-dichloro-1,3,5-triazin-2-yl)amino]benzamide are obtained in the form of a white powder and used as such in the next stage.

Fourth Stage: Preparation of the Compound of Example 4:

An intimate mixture of the product from the previous stage (0.29 g, 0.732×10⁻³ mole), neopentyl para-aminobenzalmalonate (0.5 g, 1.44×10⁻³ mole) and sodium bicarbonate (0.14 g, 1.44×10⁻³ mole) is kept in a CEM Discover microwave reactor for 4 minutes at a temperature of 60° C. and 300 watts power then for 15 minutes at a temperature of 110° C. The amorphous solid formed is extracted with dichloromethane. The organic phase is washed 3 times with water and dried then concentrated under reduced pressure. The orange oil obtained is subjected to separation on a silica column (eluent: heptane/EtOAc 75:25). Clean fractions are recovered in the form of a pale yellow oil which solidifies to give the compound of example 4 (0.6 g, yield 86%) in the form of a pale yellow powder:

UV (Ethanol): λmax=351 nm, E1%=763; λmax=298 nm, E1%=369.

### Example 5

### Synthesis of 2,4-bis-(dineopentyl 4′-aminobenzalmalonate)-6-(tert-butyl 4″-aminobenzamide)-s-triazine of Formula (5)

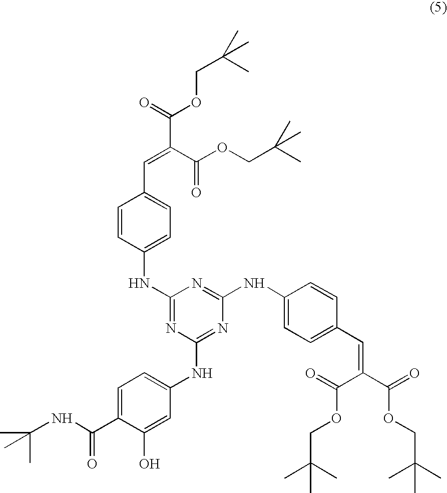

First Stage: Preparation of N-(tert-butyl)-4-nitro benzamide:

In a reactor, 4-nitrobenzoyl chloride (18.9 g, 0.1 mole) dissolved in 60 ml of methylene chloride is added in 30 minutes at a temperature of 0° C.-5° C. to a solution of tert-butylamine (8.3 g, 0.112 mole) and triethylamine (15.6 ml, 0.112 mole) dissolved in 170 ml of dichloromethane. The reaction mixture is brought back to laboratory temperature and left under stirring for 2 hours. The organic phase is washed twice with water, and dried. After removal of the solvent under reduced pressure, the solid obtained is recrystallized from isopropanol. 17.1 g (yield: 77%) of N-(tert-butyl)-4-nitro benzamide are obtained in the form of a pale yellow powder (MP 161-2° C.) and used as such in the following stage.

Second Stage: Preparation of 4-amino-N-(tert-butyl)benzamide:

In a 1 liter hydrogenator, the previous product (17.1 g, 0.077 mole) dissolved in 300 ml of isopropanol is hydrogenated in presence of 3 g of 5% palladium on charcoal as catalyst (hydrogen pressure: 7 bars) at a temperature of 60° C. for 30 minutes. After filtration, concentration of the solvent and drying under vacuum, 13.2 g (yield: 89%) of 4-amino-N-(tert-butyl)benzamide are obtained in the form of a light grey powder (MP 123-4° C.) and used as such in the following stage.

Third Stage: Preparation of N-(tert-butyl)-4-[(4,6-dichloro-1,3,5-triazin-2-yl)amino]benzamide:

In a reactor, cyanuric chloride (11.71 g, 0.06 mole) is dissolved in 150 ml of dioxane at 10° C. A solution of the product from the previous stage (11.53 g, 0.06 mole) dissolved in 60 ml of dioxane and a solution of potassium carbonate (6.3 g, 0.03 mole) dissolved in 30 ml of water are added simultaneously to this drop by drop at 10° C. in 1 hour. The heterogeneous mixture is left for 2 hours at a temperature of 10° C. About 300 ml of water are added and the precipitate formed is filtered off, then washed with water. After drying under vacuum, 18 g (yield 88%) of N-(tert-butyl)-4-[(4,6-dichloro-1,3,5-triazin-2-yl)amino]benzamide are obtained in the form of a white powder (MP 256-7° C.) and used as such in the following stage.

Fourth Stage: Preparation of the Compound of Example 5:

An intimate mixture of the previous product (1.5 g, 4.4×10⁻³ mole), dineopentyl para-aminobenzalmalonate (3 g, 8.8×10⁻³ mole) and sodium bicarbonate (0.37 g, 8.8×10⁻³ mole) is kept in a CEM Discover microwave reactor for 4 minutes at a temperature of 60° C. and 300 watts power then for 20 minutes at a temperature of 150° C. The amorphous solid formed is extracted with dichloromethane. The organic phase is washed 3 times with water and dried then concentrated under reduced pressure. The brown oil obtained is subjected to separation on a silica column (eluent: heptane/EtOAc 60:40). Clean fractions are recovered in the form of a pale yellow powder of the derivative of example 5 (0.7 g, yield 17%):

UV (Ethanol): λ=375 nm, E1%=420; λmax=345 nm, E1%=813; λ=299 nm, E1%=420.

### Example 6

### Synthesis of 2,4-bis-(1,3-dimethylbutyl 4′-aminobenzalmalonate)-6-(amyl 4″-aminobenzoate)-s-triazine of Formula (7)

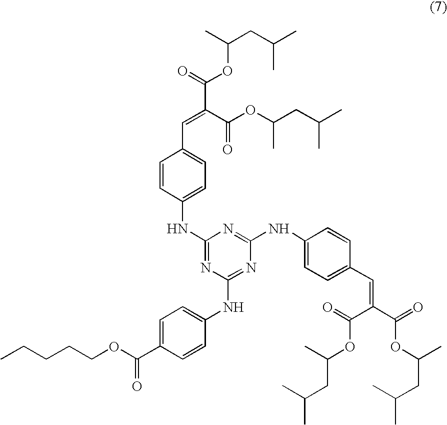

First Stage: Preparation of 1,3-dimethylbutyl Malonate:

In a reactor fitted with a Dean Stark device, malonic acid (72.8 g, 0.7 mole) and the alcohol 2-methyl-4-pentanol (286 g, 2.8 mole) are refluxed for 2 hours in 200 ml of toluene in the presence of 1.8 ml of concentrated sulfuric acid. The water formed is removed azeotropically. The organic phase is washed 3 times with water and dried over sodium sulfate. It is filtered, and the solvent evaporated. The product obtained distils at 147° C. under 20 hPa. 160 g (yield 79%) of 1,3-dimethylbutyl malonate are obtained in the form of a colorless oil which is used as such in the following stage.

Second Stage: Preparation of 1,3-dimethylbutyl 4-nitrobenzalmalonate:
p-Nitro benzaldehyde (49.9 g, 0.33 mole) and 1,3-dimethylbutyl malonate (90 g, 0.33 mole) in 150 ml of toluene are placed in a flask fitted with a Dean Stark device under a condenser and with nitrogen aeration. The catalyst prepared in advance, acetic acid (1.9 ml) and piperidine (3.3 ml) suspended in 4 ml of toluene, is added to this. The mixture is refluxed with stirring for 7 hours 30 minutes and the water formed is removed by means of the Dean Stark device. Two additions of the same quantities of catalyst were necessary. The cooled reaction mixture is poured into water and extracted with dichloromethane. The organic phase is washed with water, then dried and concentrated under reduced pressure. The red-brown oil obtained chromatographed on a silica column (eluent: heptane/EtOAc 97:3). 56.8 g (yield 43%) of clean fractions of 1,3-dimethylbutyl 4-nitrobenzalmalonate are recovered in the form of a yellow oil and used as such in the following stage.

Third Stage: Preparation of 1,3-dimethylbutyl 4-aminobenzalmalonate:

With stirring and nitrogen aeration, the derivative from the previous stage (56.8 g, 0.14 mole) is dispersed in 80 ml of acetic acid. 115 ml of water are added to this. The mixture is heated to 50° C. Iron (78.2 g, 1.4 mole) is added to this in portions without exceeding a temperature of 55° C. (addition time 1 hour). Next, acetic acid (115 ml) is added drop by drop without exceeding a temperature of 55° C. (addition time 2 hours). The mixture is heated for 1 hour more at 55° C. It is cooled, water is added and it is extracted twice with dichloromethane. The organic phase is washed with water, with a saturated solution of sodium bicarbonate, and with water, then dried over sodium sulfate. After concentration under reduced pressure, a red-brown oil is obtained which is purified by passage through a silica column (eluent: heptane/EtOAc 85:15). It is recrystallized from a mixture of heptane and 1,2-dichloroethane. 22.5 g (yield 43%) of clean fractions of 1,3-dimethylbutyl 4-aminobenzalmalonate are recovered in the form of an orange oil and used as such in the following stage.

Fourth Stage: Preparation of the Compound of Example 6:

An intimate mixture of the product from the previous stage (0.5 g, 1.32×10-3 mole), the product from the second stage of example 2 (0.235 g, 0.66×10-3 mole) and sodium bicarbonate (0.11 g, 1.32×10-3 mole) is kept in a CEM Discover microwave reactor for 4 minutes at a temperature of 60° C. and 300 watts power then for 25 minutes at a temperature of 110° C. The amorphous solid formed is extracted with dichloromethane. The organic phase is washed 3 times with water and dried, then concentrated under reduced pressure. The orange oil obtained is subjected to separation on a silica column (eluent: heptane/EtOAc 80:20). Clean fractions are recovered in the form of a yellow oil which solidifies to give the compound of example 6 (0.4 g, yield 57%) in the form of pale yellow flakes:

UV (Ethanol): λ=370 nm, E1%=500; λmax=337 nm, E1%=800; λ=300 nm, E1%=411.

### Example 7

### Synthesis of 2,4-bis-(dimenthyl 4′-aminobenzalmalonate)-6-(amyl 4″-aminobenzoate)-s-triazine of Formula (8)

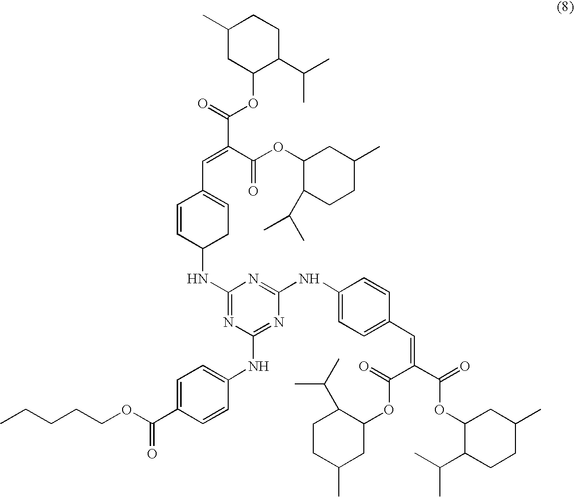

First Stage: Preparation of Dimenthyl Malonate:

In a reactor fitted with a Dean Stark device, malonic acid (22.2 g, 0.213 mole) and menthol (70 g, 0.448 mole) in 100 ml of toluene are refluxed for 6 hours in the presence of 2 ml of concentrated sulfuric acid. The water formed is removed azeotropically. The organic phase is washed 3 times with water and dried over sodium sulfate. The excess menthol is removed by vacuum distillation (140° C. at 0.6 hPa). The residue is treated with animal charcoal in refluxing isopropanol. After filtration, rinsing and evaporation of the solvent, 61.8 g (yield 76%) of menthyl malonate is obtained in the form of a yellow oil which is used as such in the following stage.

Second Stage: Preparation of 1,3-dimethylbutyl 4-nitrobenzalmalonate:

p-Nitrobenzaldehyde (21.8 g, 0.144 mole) and menthyl malonate (61 g, 0.16 mole) in 100 ml of toluene are placed in a flask fitted with a Dean Stark device under a condenser and with nitrogen aeration. The catalyst prepared in advance, acetic acid (0.92 ml) and piperidine (1.6 ml) suspended in 3 ml of toluene, is added to this. The mixture is refluxed with stirring for 9 hours and the water formed is removed by means of the Dean Stark device. Three additions of the same quantities of catalyst were necessary. The cooled reaction mixture is poured into water and extracted with dichloromethane. The organic phase is washed with water, then dried and concentrated under reduced pressure. The red-brown oil obtained is chromatographed on a silica column (eluent: heptane/EtOAc 95:5). 37 g (yield 50%) of clean fractions of menthyl 4-nitrobenzalmalonate are recovered in the form of a yellow oil and used as such in the following stage.

Third Stage: Preparation of 1,3-dimethylbutyl 4-aminobenzalmalonate:

With stirring and argon aeration, the derivative from the previous stage (37 g, 0.072 mole) is dispersed in 30 ml of acetic acid and 45 ml of water. The mixture is heated to 50° C. Iron (24.4 g, 0.437 mole) is added to this in portions without exceeding a temperature of 55° C. (addition time 30 minutes). Next, acetic acid (45 ml) is added drop by drop without exceeding a temperature of 55° C. (addition time 1 hour 30 minutes). The mixture is heated for 1 hour more at 55° C. It is cooled, water is added, and it is extracted twice with dichloromethane. The organic phase is washed with water, with a saturated solution of sodium bicarbonate, and with water, then dried over sodium sulfate. After concentration under reduced pressure, an orange gum is obtained, which is purified by passage through a silica column (eluent: heptane/EtOAc 90:10). 8.6 g (yield 25%) of clean fractions of menthyl 4-aminobenzalmalonate are recovered in the form of a yellow solid and used as such in the following stage.

Fourth Stage: Preparation of the Compound of Example 7:

An intimate mixture of the previous product (2 g, 4.1×10-3 mole), 2,4-dichloro-6-(amyl 4′-aminobenzoate)-s-triazine (first stage of example 2) (0.73 g, 2.05×10-3 mole) and sodium bicarbonate (0.34 g, 4.1×10-3 mole) is kept in a CEM Discover microwave reactor for 30 minutes at a temperature of 130-140° C. and 300 watts power. The amorphous solid formed is extracted with dichloromethane. The organic phase is washed 3 times with water and dried, then concentrated under reduced pressure. The brown oil obtained is subjected to separation on a silica column (eluent: heptane/EtOAc 80:20). Clean fractions are recovered in the form of a pale yellow powder of the compound of example 7 (0.5 g, yield 16%):

UV (Ethanol): λ=370 nm, E1%=391; λmax=340 nm, E1%=538; λ=300 nm, E1%=313.

COMPOSITION EXAMPLES 8 TO 11:

| | EX. 8 | | | |
|---|---|---|---|---|
| INGREDIENTS | (control) | EX. 9 | EX. 10 | EX. 11 |
| GLYCERYL STEARATE | 1 | 1 | 1 | 1 |
| (and) PEG-100 STEARATE | | | | |
| SIMULSOL 165 (Seppic) | | | | |
| STEARIC ACID | 1.5 | 1.5 | 1.5 | 1.5 |
| STEARINE TP 1200 | | | | |
| (STEARINERIE DUBOIS) | | | | |
| POLY DIMETHYLSILOXANE | 0.5 | 0.5 | 0.5 | 0.5 |
| 200 FLUID 350 CS (Dow | | | | |
| Corning) | | | | |
| CETYL ALCOHOL | 0.5 | 0.5 | 0.5 | 0.5 |
| NACOL 16-98 (SASOL) | | | | |
| CETEARYL ALCOHOL | 2 | 2 | 2 | 2 |
| (AND) CETEARYL | | | | |
| GLUCOSIDE | | | | |
| MONTANOV 68 (SEPPIC) | | | | |
| PRESERVATIVE | 1 | 1 | 1 | 1 |
| TRIETHANOLAMINE | 0.45 | 0.45 | 0.45 | 0.45 |
| C12-15 ALKYL BENZOATE | 15 | 15 | 15 | 15 |
| FINSOLV TN (WITCO) | | | | |
| TRIAZINE | — | 1 | 2 | 4 |
| 1,3,5/BENZALMALONATE | | | | |
| (EXAMPLE 1) | | | | |
| OCTYL METHOXY | 5 | 5 | 5 | 5 |
| CINNAMATE PARSOL MCX | | | | |
| (DSM) | | | | |
| GLYCERINE | 5 | 5 | 5 | 5 |
| PRICERINE 9091 | | | | |
| (UNIQUEMA) | | | | |
| XANTHAN GUM | 0.2 | 0.2 | 0.2 | 0.2 |
| KELTROL T | | | | |
| (NUTRASWEET) | | | | |
| ETHYLENE DIAMINE | 0.1 | 0.1 | 0.1 | 0.1 |
| TETRAACETIC ACID, | | | | |
| DISODIUM SALT, 2 H2O | | | | |
| (VERSENE NA2) | | | | |
| WATER | 67 | 67 | 66 | 64 |
| POTASSIUM CETYL | 1 | 1 | 1 | 1 |
| PHOSPHATE | | | | |
| AMPHISOL K (Givaudan) | | | | |
| ACRYLATES/C10-30 ALKYL | 0.2 | 0.2 | 0.2 | 0.2 |
| ACRYLATE | | | | |
| CROSSPOLYMER | | | | |
| PEMULEN TR-1 (NOVEON) | | | | |
| ISOHEXADECANE | 1 | 1 | 1 | 1 |
| ISOHEXADECANE (BP) | | | | |
| TRIETHANOLAMINE | qs pH | qs PH | qs pH | qs pH |

Method for Measurement of Photostability:

For each formula, constituents given in % by weight, 3 test samples and 3 control samples were prepared. Using a spatula, 2 mg/cm2 of formula are deposited on polymethyl methacrylate plates.

The test plates are exposed for 38 minutes in the HERAUS SUN TEST equipped with a Xenon lamp having a UV-A flux of 9.51×10⁻³ W/cm2 and a UV-B flux of 5.43×10-4 W/cm2. The control plates are stored in darkness for the same time and at the same temperature (38-40° C.).

At the end of this time, the extraction of the filters is effected by immersing each plate in 50 g of methanol and subjecting them to ultrasound for 15 mins to ensure good extraction. The solutions obtained are analyzed by HPLC and UV spectrophotometry.

For each formula tested, the proportion of 4-tert.-butyl-4′-methoxy-dibenzoylmethane remaining after exposure is given as the ratio of its optical density (OD) in the exposed sample to its unexposed optical density (OD). These are based on the absorption maximum corresponding to butyl-methoxy-dibenzoylmethane: λmax=358 nm.

The results obtained are summarized in the Table below:

TABLE

| | Remaining % of cinnamate |
|---|---|
| Compositions | after 1 hour UV-A exposure |
| Formula 8 | 60 |
| (control) | |
| Formula 9 | 66 |
| Formula 10 | 70 |
| Formula 11 | 79 |

An improvement in the photostability of the cinnamate ester is clearly seen in the presence of the compound of Example 1.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable, photostable sunscreen composition, comprising at least one cinnamate ester UV-B filter and at least one s-triazine compound having the following formula (I): 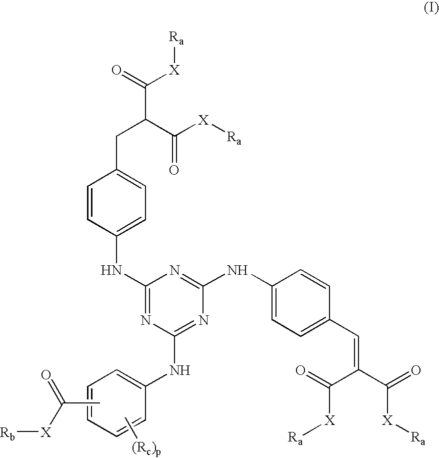
in which:
the groups X, which may be identical or different, are each —O— or —NR₆—;
the radicals Rₐ, which may be identical or different, are each a radical of formula (II): 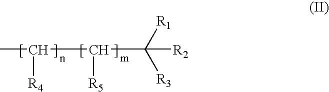
in which:
the radicals R₁ and R₂, which may be identical or different, are each a linear or branched C₁-C₈ alkyl radical, with the proviso that R₁ and R₂ may together form a C₅-C₈ ring member, optionally substituted by 1, 2 or 3 linear or branched C₁-C₄ alkyl radicals;
the radicals R₃, R₄ and R₅, which may be identical or different, are each a hydrogen atom or a linear or branched C₁-C₄ alkyl radical;
n has the value 0 or 1;
m has the value 0 or 1;
with the provisos that:
(i) when n=1 and R₄ is hydrogen, then m is equal to 0 and R₃ is other than hydrogen;
(ii) when R₁ and R₂ together form a C₅-C₈ ring member, then the sum n+m is other than 2;
R₆ is hydrogen or a C₁-C₈ alkyl radical;
R_{b} is a linear or branched and optionally unsaturated C₁-C₂₀ alkyl radical, a C₅-C₁₂ cycloalkyl radical optionally substituted by 1 to 3 linear or branched C₁-C₄ alkyl radicals, the —(CH₂CHR₇—O)_{q}R₈ radical or the —CH₂—CH(OH)—CH₂—O—R₈ radical;
R₇ is hydrogen or methyl;
R₈ is hydrogen or a C₁-C₈ alkyl radical;
q=1-20,
the COXR_{b} group can be in the ortho, meta or para position with respect to the amino group;
R_{c} is a saturated or unsaturated and linear or branched C₁-C₂₀ alkyl radical, the OH group or a linear or branched C₁-C₂₀ alkoxy radical; and
p is equal to 0, 1 or 2, formulated into a topically applicable, cosmetically acceptable medium therefor.

2. The photostable sunscreen composition as defined by claim 1, wherein formula (1):
(a) n=m=0 and
(b) R₁, R₂, R₃ are each a C₁-C₄ alkyl radical or R₃ is hydrogen and R₁ and R₂ together form a C₅-C₈ ring member optionally substituted by one or two alkyl radicals.

3. The photostable sunscreen composition as defined by claim 1, wherein formula (1):
(a) n=1 and R₄ is an alkyl radical or m=1 and R₅ is an alkyl radical, and
(b) R₁ and R₂ are each a C₁-C₄ alkyl radical.

4. The photostable sunscreen composition as defined by claim 1, wherein the compounds of formula (I) are selected from the group consisting of the compounds of following formulae (1) to (10):

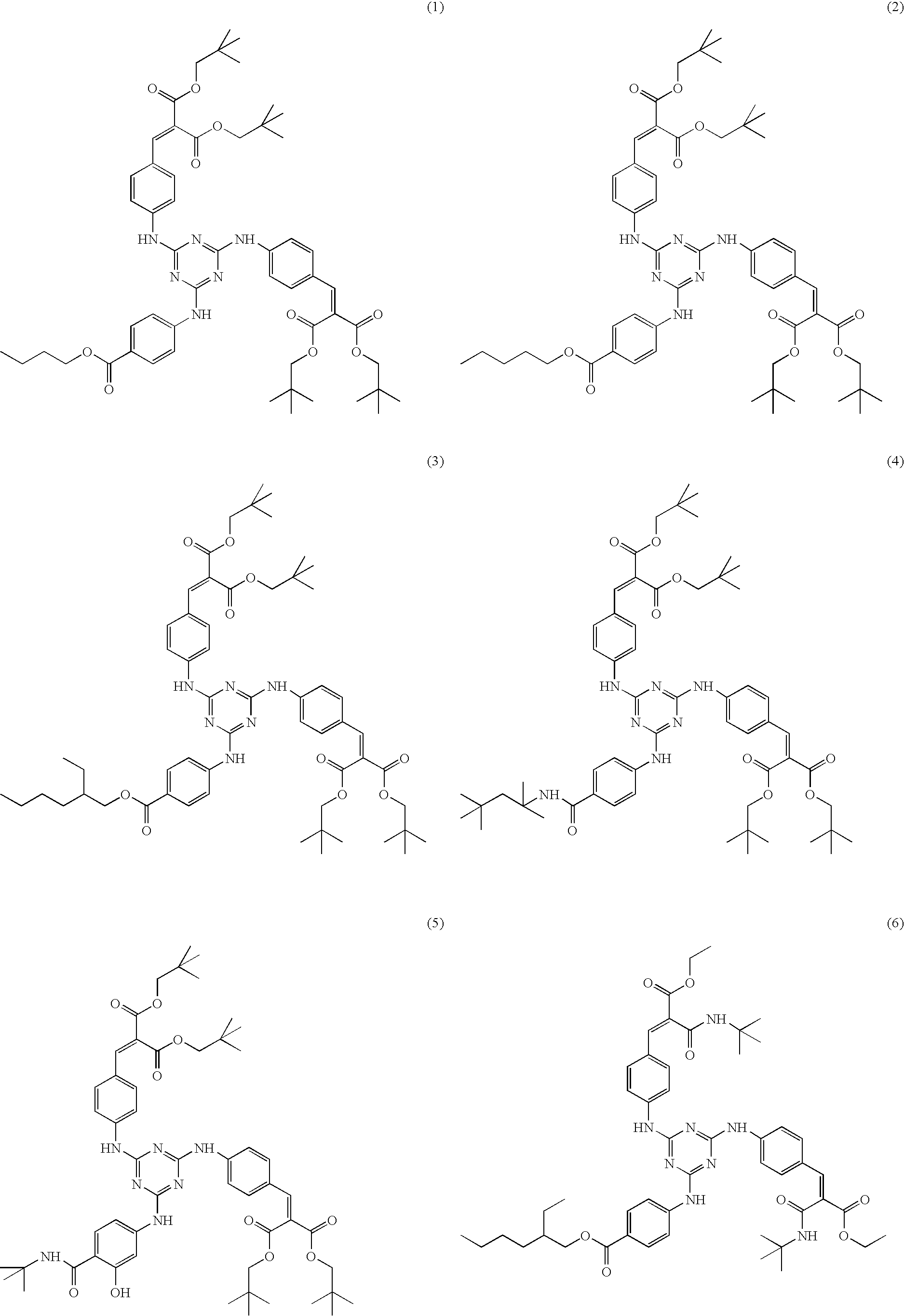

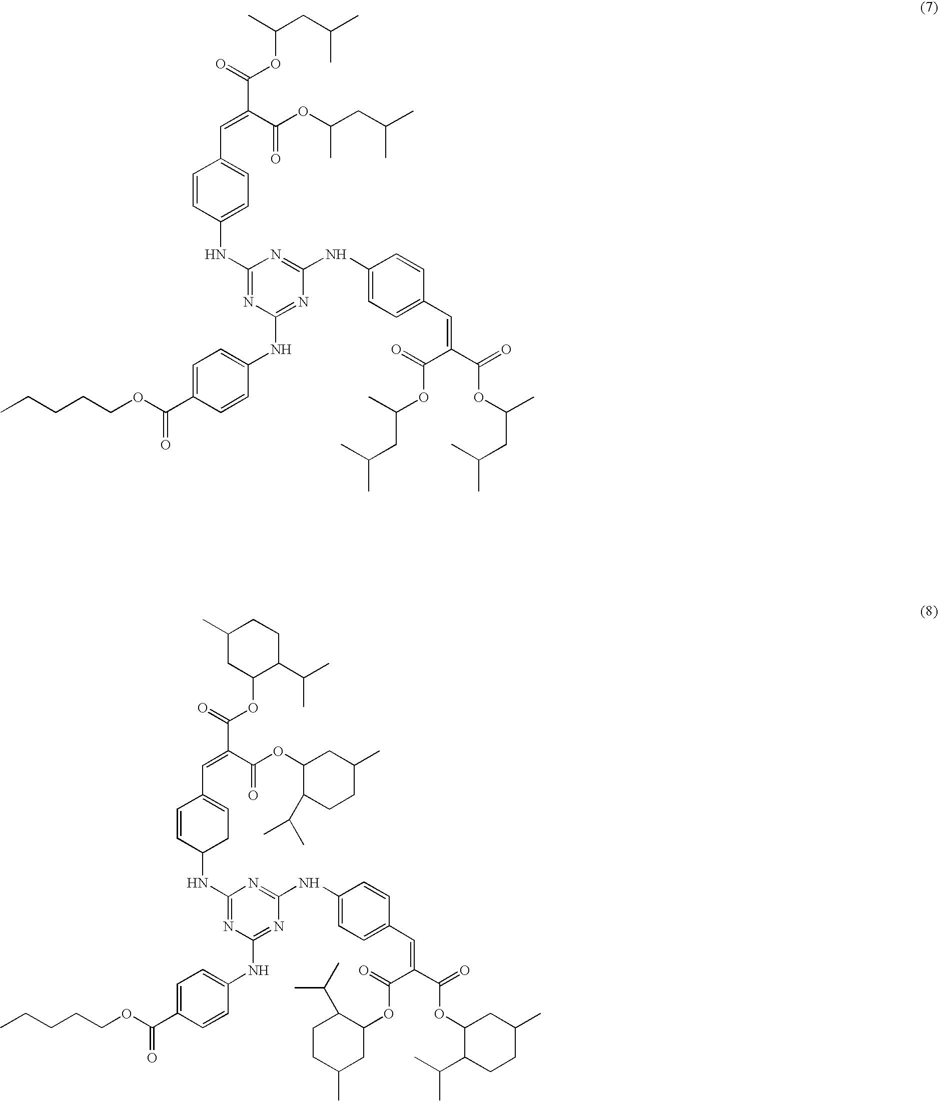

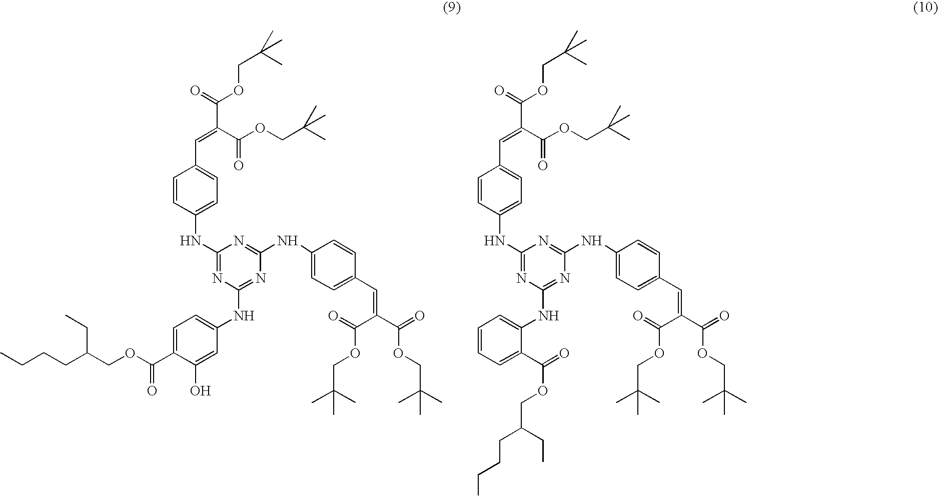

5. The photostable sunscreen composition as defined by claim 4, wherein the compound of formula (1) comprises 2,4-bis(dineopentyl 4′-aminobenzalmalonate)-6-(butyl 4″-aminobenzoate)-s-triazine of formula (1).

6. The photostable sunscreen composition as defined by claim 1, said at least one cinnamic acid ester UV-B filter having the following formula (A): 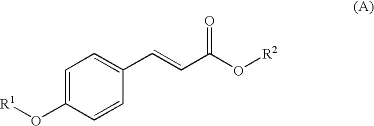
in which:
the radicals R¹ and R², which may be identical or different, are each a linear or branched C₁-C₂₄ alkyl radical.

7. The photostable sunscreen composition as defined by claim 6, said at least one cinnamic acid ester UV-B filter being selected from the group consisting of:
Ethylhexyl Methoxycinnamate or Cinoxate,
Isopropyl Methoxycinnamate,
Isoamyl Methoxycinnamate, and
Diisopropyl Methylcinnamate.

8. The photostable sunscreen composition as defined by claim 7, said at least one cinnamic acid ester UV-B filter comprising Ethylhexyl Methoxycinnamate or Cinoxate.

9. The photostable sunscreen composition as defined by claim 1, said at least one cinnamic acid ester UV-B filter comprising from 0.01% to 20% by weight thereof, with respect to the total weight of the composition.

10. The photostable sunscreen composition as defined by claim 1, said at least one compound of formula (I) comprising from 0.01% to 20% by weight thereof, with respect to the total weight of the composition.

11. The photostable sunscreen composition as defined by claim 1, further comprising at least one other organic or inorganic photoprotective agent active in the UV-A and/or UV-B region which is water-soluble or fat-soluble or insoluble in the conventional cosmetic solvents.

12. The photostable sunscreen composition as defined by claim 11, comprising at least one additional organic photoprotective agent selected from the group consisting of anthranilates; salicylic derivatives; camphor derivatives; benzophenone derivatives; β,β-diphenylacrylate derivatives; triazine derivatives other than those of formula (I); benzotriazole derivatives; benzalmalonate derivatives other than those of formula (I); benzimidazole derivatives; imidazolines; bis-benzoazolyl derivatives; p-aminobenzoic acid (PABA) derivatives other than those of formula (I); methylenebis(hydroxyphenylbenzotriazole) derivatives; benzoxazole derivatives; screening polymers and screening silicones; dimers derived from α-alkylstyrene; 4,4-diarylbutadienes; and mixtures thereof.

13. The photostable sunscreen composition as defined by claim 12, comprising at least one of the following compounds:
Ethylhexyl Salicylate,
Homosalate,
Octocrylene,
Phenylbenzimidazole Sulfonic Acid,
Disodium Phenyl Dibenzimidazole Tetrasulfonate,
Benzophenone-3,
Benzophenone-4,
Benzophenone-5,
Diethylamino Hydroxybenzoyl Hexyl Benzoate,
4-Methylbenzylidene Camphor,
Terephthalylidene Dicamphor Sulfonic Acid,
Bis-Ethylhexyloxyphenol Methoxyphenyl Triazine,
Ethylhexyl Triazone,
Diethylhexyl Butamido Triazone,
Methylene Bis-Benzotriazolyl Tetramethylbutylphenol,
Drometrizole Trisiloxane,
Polysilicone-15,
1,1-Dicarboxy (2,2′-Dimethylpropyl)-4,4-Diphenylbutadiene, and
and mixtures thereof.

14. The photostable sunscreen composition as defined by claim 11, comprising at least one additional inorganic photoprotective agent selected from the group consisting of pigments or nanopigments formed of metal oxides, whether treated or untreated.

15. The photostable sunscreen composition as defined by claim 14, comprising pigments or nanopigments of titanium, zinc, iron, zirconium or cerium oxides and mixtures thereof, whether treated or untreated.

16. The photostable sunscreen composition as defined by claim 1, further comprising at least one agent for the artificial tanning and/or browning of the skin.

17. The photostable sunscreen composition as defined by claim 1, further comprising at least one adjuvant selected from the group consisting of fatty substances, organic solvents, ionic or nonionic and hydrophilic or lipophilic thickeners, softening agents, humectants, opacifiers, stabilizing agents, emollients, silicones, anti-foaming agents, fragrances, preservatives, anionic, cationic, nonionic, zwitterionic or amphoteric surfactants, active principles, fillers, polymers, propellants, basifying or acidifying agents, and mixtures thereof.

18. The photostable sunscreen composition as defined by claim 1, formulated as an oil-in-water or water-in-oil emulsion.

19. The photostable sunscreen composition as defined by claim 1, formulated as a product for the cosmetic treatment of the skin, lips, nails, hair, eyelashes, eyebrows and/or scalp.

20. The photostable sunscreen composition as defined by claim 1, formulated as a care product for the skin, lips, nails, hair and/or scalp.

21. The photostable sunscreen composition as defined by claim 1, formulated as a makeup product.

22. A method for improving the chemical stability with respect to UV radiation of at least one cinnamic acid ester UV-B filter which comprises combining therewith at least one s-triazine compound of formula (1) as defined in claim 1.

23. A method of improving the effectiveness against UV-B rays of at least one cinnamic acid ester UV-B filter, comprising combining therewith at least one s-triazine compound of formula (I) as defined in claim 1.

24. A regime or regimen for photoprotecting the skin, lips, nails, hair, eyelashes, eyebrows, and/or scalp against the damaging effects of UV radiation, comprising topically applying thereon a thus effective amount of the photostable sunscreen composition as defined in claim 1.
